(19) United States
(12) Patent Application Publication
(10) Pub. No.: US 2011/0277796 A1
(43) Pub. Date: Nov. 17, 2011

(54) METHOD OF CLEANSING SKIN HAVING AN IMPAIRED BARRIER

(76) Inventors: Russel Walters, Philadelphia, PA (US); Neena Tierney, Yardley, PA (US); Lisa Gandolfi, Franklin Park, NJ (US); Donzel Johnson, Morrisville, PA (US); Katharine M. Martin, Ringoes, NJ (US)

(21) Appl. No.: 12/779,211

(22) Filed: May 13, 2010

(51) Int. Cl. A61K 8/81; B08B 3/04; A61Q 19/10

(52) U.S. Cl. 134/26; 510/130

(57) ABSTRACT

This invention relates to a method of cleansing skin having an impaired barrier function. The method of this invention utilizes cleansing compositions that have the characteristic of minimally perturbing or interacting with the skin barrier by applying to the skin a cleansing composition comprising (i) at least one low molecular weight, non-cross-linked, linear acrylic copolymer and (ii) at least one surfactant selected from the group consisting of anionic surfactants, amphoteric surfactants, and combinations of two or more thereof, the cleansing composition having a ΔST(65) of greater than about 2 mN/m.

### FIELD OF THE INVENTION

This invention relates to a method of cleansing skin having an impaired barrier function. The method of this invention utilizes cleansing compositions that have the characteristic of minimally perturbing or interacting with the skin barrier. This characteristic is essential in order not to exacerbate a skin condition which has caused the barrier function impairment.

### BACKGROUND OF THE INVENTION

Human skin acts as a barrier and protective layer against chemical, physical and mechanical insults. The primary function of the skin's outermost epidermal layer, the stratum corneum (SC), is preventing the loss of water and vital substances from the body into the environment while keeping noxious exogenous materials from penetrating the skin barrier to living tissue. Other functions of the SC include regulating homeostatic mechanisms to assure steady-state conditions in the face of a changing environment and providing mechanisms of repair if the barrier is disrupted by disease or exogenous insults or the presence of anti-bacterial substances to protect against infection. In order to fulfill these functions, the SC needs to be hydrated and flexible.

There are a multitude of factors, including disease, diet, race, as well as the external environment, that may render the skin barrier more prone to perturbation and potentially induce dryness, irritation, or itch. As individuals age, their ability to restore the barrier is diminished. Psychological stress may lead to elevated levels of circulating glucocorticoids, which also delays barrier recovery. Seasonal variation in intercellular lipid levels may also explain the predisposition of skin to dryness in the winter months.

Thus, complicated mechanisms in the skin maintain the essential hydration state of the SC. However, many factors may work together to compromise the stratum corneum (hereinafter, “SC”) barrier function and increase the rate of water loss of the skin. Exposure to harsh conditions, including cold, dry winter weather, frequent washing with soap and hot water, exposure to surfactants or irritating chemicals or solvents may compromise the SC barrier function causing skin dryness. Diaper rash may also compromise the SC barrier. Apart from external factors provoking an impaired SC barrier function, several diseases are known that are characterized by an abnormal condition or behavior of the SC. Examples of such diseases include various forms of dermatitis, including atopic dermatitis, psoriasis, ichthyosis, eczema, seborrhea, hand dermatitis, poison ivy, diaper rash, surfactant-induced xerosis, soap-induced xerosis, winter-induced xerosis, and clinically diagnosed dry skin.

Skin having an impaired stratum corneum barrier function usually presents as an elevated transepidermal water loss (TEWL) or decreased skin hydration. Skin with an impaired barrier is generally treated by applying moisturizers, anti-inflammatory agents, steroids, and the like. In the case of diaper rash, skin protectants such as zinc oxide and petrolatum are often applied.

Cleansing the skin may also disrupt the skin barrier or aggravate impaired barrier function that is already present in the skin. Consumers suffering from impaired barrier function typically cleanse with water alone, use emulsion based cleansers (surfactant free), or reduce the frequency of skin cleansing in order to avoid exacerbating the condition.

Thus, it is desirable to create a method for cleansing impaired skin that does not exacerbate the impaired skin barrier function.

### BRIEF DESCRIPTION OF THE DRAWINGS

FIG. 1 is a graph illustrating Dynamic surface tension, [γ(t)−γ_{eq}]. [γ(t)−γ_{eq}], as defined below.

FIG. 2 is a graph illustrating ΔST(t) as a function of time, the difference between the dynamic surface tension of the cleanser with hydrophobically-modified polymer and the corresponding cleanser without hydrophobically-modified polymer.

### SUMMARY OF THE INVENTION

The methods of this invention relate to cleansing skin that has an impaired barrier in such a way that the barrier does not become further impaired. More particularly, the methods of this invention relate to the use of cleansing compositions having slower surface dynamic characteristics to cleanse skin having an impaired barrier so as not to further impair the barrier. The content of surfactants in the compositions useful in the methods of this invention is relatively low compared with other cleansing compositions, yet the compositions produce a foam when used in the cleansing process.

### DETAILED DESCRIPTION OF THE INVENTION

As used herein, the phrase “impaired skin barrier” relates to those skin conditions wherein the stratum corneum has been compromised. The impaired skin barrier function can be caused by acute or chronic irritation to the skin via external aggressors, skin disease, stress and the like. Included in this definition are conditions such as various forms of dermatitis, including atopic dermatitis, psoriasis and ichthyosis, eczema, seborrhea, hand dermatitis, poison ivy, diaper rash, surfactant-induced xerosis, soap-induced xerosis, winter-induced xerosis, and clinically diagnosed dry skin.

Premature, as well as full-term, newborn babies also have thin or low-functioning stratum corneum and, in fact, the stratum corneum barrier function of infants continues to develop at least for the first twelve months following birth, and may also continue to develop over the first several years of life.

As used herein, the term “foaming cleansing composition” includes those compositions that have the ability to remove lipids, oils and natural components from the skin surface and which produce a foam (i.e., a system of bubbles surrounded by film). A cleansing composition is typically applied to the skin and rinsed off with water. Rubbing with the fingers, hands or wash cloth or pouring into a bath may result in sudsing or foaming of the cleanser. If the skin has an impaired barrier prior to cleansing and exposure to foaming cleansing composition, certain types of cleansing compositions can be further damaging to the health and integrity of the skin barrier already in distress. In particular, cleansing compositions containing arelatively high surfactant content will tend to be more damaging to the skin barrier function.

In particular, skin cleansing formulations contain surfactants that emulsify soils on the skin surface for removal with a water rinse. Surfactants may be anionic, cationic, nonionic, or zwitterionic and can be in the form of a bar, a liquid, a cream, a gel or the like. Surfactants vary markedly in their effects on the skin and differ significantly in their effect on the skin barrier. They have been shown to vary in their effects on corneocyte swelling, disaggregation, and damage. Surfactants, as well as other topical treatments, can vary greatly in their effects on the permeability barrier of skin.

As used herein the term “low molecular weight” polymer refers to a polymer having a number average molecular weight (Mₙ) as measured by gel permeation chromatography (GPC) calibrated with a poly(methyl methacrylate) (PMMA) standard of about 100,000 or less. In certain preferred embodiments, low-molecular weight polymers are those having molecular weight ranges of from about 5,000 to about 80,000 Mₙ, more preferably from about 10,000 to about 50,000 Mₙ, and more preferably between about 15,000 and 40,000 Mₙ.

### Measuring the Impairment of Barrier Function

TEWL and skin hydration constitute two areas of measurements by which to determine whether skin barrier has been impaired. However, absolute measurements generated by these test methods may require additional means by which to understand the characteristics and extent of barrier impairment. For example, two different people may be exposed to the environment but present very different TEWL or skin hydration measurements from their exposed skin depending on the nature of their own particular skin properties. Likewise, different environments may produce similar TEWL or skin hydration measurements in very different people. Therefore, when determining the effect of the application of the compositions of this invention to skin having impaired barrier function, it is best to examine how the TEWL or skin hydration level may change upon exposure to cleansing compositions and measure the change in TEWL or skin hydration after exposure. In addition, TEWL and skin hydration may be linked to surfactant kinetics and dynamics.

It has been demonstrated that impaired skin barrier may have certain physical characteristics, including a higher TEWL, although decreased hydration level is not always initially present in skin having impaired barrier function. However, it is desirable, when cleansing skin with impaired barrier function, not to increase the transepidermal water loss and thereby cause additional impairment.

The hydration level of the stratum corneum affects its mechanical and electrical properties, thus the Ski-Con-200EX (I.B.S Co., LTD., Japan), which measures the high frequency conductivity of the skin, can be used to measure the relative water holding capacity of the superficial corneocytes (first layer). The measurement may be performed by placing a probe on the skin surface for a period of time. The probe is connected to a computer or other data recording device. Skin hydration measured via conductance is expressed as micro Siemans, “μS”.

### Surfactant Kinetics

There are a number of different ways in which a cleansing composition may affect an impaired barrier. Different theories exist as to whether surfactant existing as monomer or surfactant existing as micelle is responsible for surfactant penetration into the skin.

The classical monomer penetration model of surfactant penetration holds that only surfactant monomers, being of relatively small size, are able to penetrate into the SC. According to the model, at concentrations above the critical micelle concentration (CMC), surfactants that have self-assembled into micelles are too large to penetrate into the SC. Thus, for surfactant systems exhibiting low values of CMC, there will be fewer monomeric surfactants available to penetrate into the skin.

As set forth in U.S. Pat. Nos. 6,468,614 and 6,703,427, surfactant that exists as monomer is critical in damaging the skin and therefore teaches the addition of cosurfactant to a cleansing composition. The cosurfactant results in a reduced critical micelle concentration (CMC) relative to the cleansing composition without cosurfactant. A reduced CMC suggests a reduced concentration of surfactant that exists as monomer.

Surfactant solutions are highly dynamic: monomeric surfactants rapidly hop into and out of micelles with exchange rates on the order of milliseconds to seconds, and entire micelles form and dissociate on the order of seconds. As surfactant rapidly exchanges between monomer and micelle, likely both surfactant monomer and surfactant in micelle are responsible for damage to the skin barrier. Surfactant that exists as monomer will soon be in a micelle and likewise, surfactant that exists in the micelle will rapidly hop out of the micelle and become monomer.

The surfactant in a cleanser during use undergoes rapid dilution from the initial, “in the bottle” concentration (where most of the surfactant exist in micelles) to the more dilute “in use” concentrations, typically 1-10% of the initial concentrations. During this dilution, surfactant is predominately moving from micelle to monomer. It is clear that micellar surfactant contributes to penetration, either by direct micellar penetration or by acting as a reservoir to provide a continual source of monomeric penetrating species.

One way in which to measure the effect of a solution on the skin barrier, in accordance with the methods of this invention, is to examine the characteristics of the solution being placed on the skin. In particular, when using a surfactant system, it is important to understand the kinetics of the surfactant(s) present in the system. For example, in a composition containing surfactants, an important measurement is that of the speed at which the surfactant arrives at the water/skin interface. Surfactants present in a surfactant composition form micelles, or aggregate structures having the hydrophilic “head” regions of the surfactant molecules in contact with surrounding solvent, and in which the hydrophobic single “tail” regions are sequestered in the micelle centre. When a new interface is formed, the surface is initially largely devoid of surfactant and the surface tension is high, similar to that of pure water. With time, the surfactant in the solution moves to the outer surface-interface, where it will interact with the air or skin, which results in a reduction of surface tension. Dynamics of surfactant self-assemblies: micelles, microemulsions, vesicles and lyotropic phases, Edited Raoul Zana, CRC Press, Boca Raton, Fla., Vol. 125, 2005

We have found that slower surface dynamics result in a lower tendency to cause an increase in skin barrier impairment. By understanding the kinetics of the surfactant compositions, one can design compositions with a lower energy state and slower dynamics. This results in compositions having a lower tendency to cause additional impairment to skin already possessing barrier impairment.

Ways of measuring surfactant dynamics include drop shape analysis (DSA) and bubble pressure tensiometry (BPT). Both DSA and BPT measure the surface tension as a function of time in which a solution forms a new interface between the aqueous phase and air. In both DSA and BPT, the surfactant initially exists in the bulk of the aqueous phase. Once a new interface is formed, the surfactant then diffuses toward the new interface and begins to populate the air/water interface, thereby reducing the surface tension over time. The decrease in the surface tension (measured in milliNewtons per Meter, or mN/M) over time results from the surfactant populating the newly formed interface.

In the compositions of this invention, the addition of a low molecular weight hydrophobically-modified polymer (“hm-polymer”) allows for a lower energy state to exist in the aqueous composition, resulting in slower dynamics. The surfactant associated to the low molecular weight hm-polymer is more stable than the surfactant that exists as a micelle. Thus, surfactant in a micelle more readily hops out of the micelle, or the micelle breakdown more rapidly than surfactant associated with low molecular weight hm-polymer.

As used herein, the term Delta surface tension (“ΔST”) means the difference between the ST of the solution containing the preferred surfactant less the ST of the same solution without the preferred surfactant (surfactant and low-molecular weight hm-polymer).

Delta ST is measured at a total surfactant concentration of 288 mg/L, and under the experimental parameters for the dynamics surface tension test and the equilibrium tensiometry test as defined above.

γ(t)=surface tension a time t

γ(65); surface tension at time=65 s (seconds)

γ_{eq}=equilibrium surface tension, surface tension at very long times, determined by Wilhemy plate tensiometry; the equilibrium tensiometry test.

γ(65)−γ_{eq}; dynamic surface tension, difference in the surface tension at time=65 s from the equilibrium surface tension, as determined by Wilhemy plate tensiometry.

ΔST (65)=[γ(65)−γ_{eq}]_{with hm-polymer} −[γ(65)−γ_{eq}]_{without hm-polymer}; difference in the dynamic surface tension between cleansing with a low molecular weight non cross-linked hm-polymer at time of 65, and a cleansing solution without hm-polymer.

In assessing the dynamic nature of surfactant solutions, it is most relevant to determine the difference between the surface tension as a function of time and the surface tension at equilibrium (i.e. the surface tension at long times), [γ(t)−γ_{eq}]. Theoretical approaches to surfactant dynamics attempt to explain this difference between the surface tension as a function of time and the surface tension at equilibrium [γ(t)−γ_{eq}] based on the transport of surfactant to the air/water interface as a function of time.

We have discovered that, surprisingly, it is possible to cleanse skin having impaired barrier function using compositions that are mild to the skin and which only minimally change the impairment of the skin barrier and yet are able to produce a foam.

The methods of this invention comprise, consist essentially of and consist of applying a composition containing (a) at least one surfactant and (b) a low molecular weight hydrophobically-modified polymer capable of binding a surfactant to skin having an impaired barrier function and rinsing said composition from the skin, whereby the composition does not exacerbate the impaired barrier function and yet the composition may preferably be capable of producing foam.

The addition of a low molecular weight hydrophobically-modified polymer to the cleansing compositions of this invention allows a lower energy state to exist in the aqueous composition, resulting in slower surfactant dynamics.

Although applicants do not wish to be bound by or to any particular theory of operation, it is believed that surfactant associated with the low molecular weight hydrophobically-modified polymer is more stable than surfactants that exist as a micelle. Thus, surfactant contained in a micelle structure more readily disperses out of the micelle than it does when associated with low molecular weight hydrophobically-modified-polymer.

The polymeric material useful in the methods of this invention is preferably a composition suitable for associating anionic and/or amphoteric surfactant thereto and is a non-crosslinked, linear acrylic copolymer that mitigates the impaired dermal barrier damage typically associated with surfactant systems without substantially increasing viscosity build. The non-crosslinked, linear polymers are preferably of low molecular weight having a number average molecular weight of 100,000 or less as measured by gel permeation chromatography (GPC) calibrated with a poly(methyl methacrylate) (PMMA) standard (as used herein, unless otherwise specified, all number average molecular weights (Mₙ) refer to molecular weight measured in such manner). The copolymeric mitigant is polymerized from at least two monomeric components. The first monomeric component is selected from one or more α,β-ethylenically unsaturated monomers containing at least one carboxylic acid group. This acid group can be derived from monoacids or diacids, anhydrides of dicarboxylic acids, monoesters of diacids, and salts thereof. The second monomeric component is hydrophobically modified (relative to the first monomeric component) and is selected from one or more α,β-ethylenically unsaturated non-acid monomers containing a C₁ to C₉ alkyl group, including linear and branched C₁ to C₉ alkyl esters of (meth)acrylic acid, vinyl esters of linear and branched C₁ to C₁₀ carboxylic acids, and mixtures thereof. In one aspect of the invention the second monomeric component is represented by the formula:

CH₂═CRX

wherein R is hydrogen or methyl; X is —C(O)OR′ or —OC(O)R²; R¹ is linear or branched C₁ to C₉ alkyl; and R² is hydrogen or linear or branched C₁ to C₉ alkyl. In another aspect of the invention R¹ and R² is linear or branched C₁ to C₈ alkyl and in a further aspect R¹ and R² are linear or branched C₂ to C₅ alkyl.

Exemplary first monomeric components include (meth)acrylic acid, itaconic acid, citraconic acid, maleic acid, fumaric acid, crotonic acid, aconitic acid, and mixtures thereof. Exemplary second monomeric components include ethyl (meth)acrylate, butyl (meth)acrylate, 2-ethylhexyl (meth)acrylate, vinyl formate, vinyl acetate, 1-methylvinyl acetate, vinyl propionate, vinyl butyrate, vinyl 2-ethylhexanoate, vinyl pivalate, vinyl neodecanoate, and mixtures thereof. As used herein, the term “(meth)acrylic” acid and “(meth)acrylate” are meant to include the corresponding methyl derivatives of acrylic acid and the corresponding alkyl acrylate For example, “(meth)acrylic” acid refers to acrylic acid and/or methacrylic acid and “(meth)acrylate” refers to alkyl acrylate and/or alkyl methacrylate.

The non-crosslinked, linear acrylic copolymer mitigants of the invention can be synthesized via free radical polymerization techniques known in the art. In one aspect of the invention, the amount of the first monomeric component to the second monomeric component utilized ranges from about 20:80 wt. % to about 50:50 wt. %, based on the total weight of all of the monomers in the polymerization medium. In another aspect the weight ratio of the first monomeric component to the second monomeric component is about 35:65 wt. %, and in a further aspect the weight ratio of first monomeric component to second monomeric component is about 25:75 wt. %, all based on the total weight of all monomers in the polymerization medium.

In another aspect emulsion polymerization techniques can be used to synthesize the non-crosslinked, linear acrylic copolymer mitigants of the invention. In a typical emulsion polymerization, a mixture of the disclosed monomers is added with mixing agitation to a solution of emulsifying surfactant, such as, for example, an anionic surfactant (e.g., fatty alcohol sulfates or alkyl sulfonates), in a suitable amount of water, in a suitable reactor, to prepare a monomer emulsion. The emulsion is deoxygenated by any convenient method, such as by sparging with nitrogen, and then a polymerization reaction is initiated by adding a polymerization catalyst (initiator) such as sodium persulfate, or any other suitable addition polymerization catalyst, as is well known in the emulsion polymerization art. The polymerization medium is agitated until the polymerization is complete, typically for a time in the range of about 4 to about 16 hours. The monomer emulsion can be heated to a temperature in the range of about 70 to about 95° C. prior to addition of the initiator, if desired. Unreacted monomer can be eliminated by addition of more catalyst, as is well known in the emulsion polymerization art. The resulting polymer emulsion product can then be discharged from the reactor and packaged for storage or use. Optionally, the pH or other physical and chemical characteristics of the emulsion can be adjusted prior to discharge from the reactor. Typically, the product emulsion has a total solids content in the range of about 10 to about 50 wt. %. Typically, the total polymer content (polymer solids) of the product emulsion is in the range of about 15 to about 45 wt. %, generally not more than about 35 wt. %.

In one aspect, the number average molecular weight (Mₙ) of the linear copolymeric mitigants of the present invention as measured by gel permeation chromatography (GPC) calibrated with a poly(methyl methacrylate) (PMMA) standard is 100,000 or less. In another aspect of the invention, the molecular weight ranges between about 5,000 and about 80,000 Mₙ, in a further aspect between about 10,000 and 50,000 Mₙ, and in a still further aspect between about 15,000 and 40,000 Mₙ.

In one aspect of the invention, the linear copolymeric mitigants have a viscosity of 500 mPa·s or less (Brookfield RVT, 20 rpm, spindle no. 1) at a 5 wt. % polymer solids concentration in deionized water and neutralized to pH 7 with an 18 wt. % NaOH solution. The viscosity can range from about 1 to about 500 mPa·s in another aspect, from about 10 to about 250 mPa·s in a further aspect, and from about 15 to about 150 mPa·s in a still further aspect.

Any of a variety of anionic surfactants may be combined with a polymeric material of the present invention to form a cleansing composition according to preferred embodiments of the present methods. According to certain embodiments, suitable anionic surfactants include those selected from the following classes of surfactants: alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl taurates, fatty alkyl sulfoacetates, alkyl phosphates, and mixtures of two or more thereof. Examples of certain preferred anionic surfactants include:

alkyl sulfates of the formula

R′—CH₂OSO₃X′;

alkyl ether sulfates of the formula

R′(OCH₂CH₂)ᵥOSO₃X′;

alkyl monoglyceryl ether sulfates of the formula

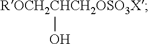

alkyl monoglyceride sulfates of the formula

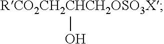

alkyl monoglyceride sulfonates of the formula

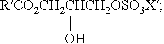

alkyl sulfonates of the formula

R′—SO₃X′;

alkylaryl sulfonates of the formula

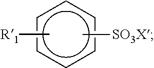

alkyl sulfosuccinates of the formula:

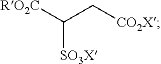

alkyl ether sulfosuccinates of the formula:

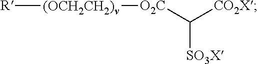

alkyl sulfosuccinamates of the formula:

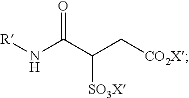

alkyl amidosulfosuccinates of the formula

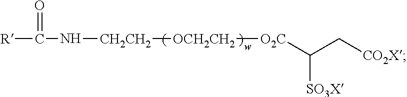

alkyl carboxylates of the formula:

R′—(OCH₂CH₂)_{w}—OCH₂CO₂X′; alkyl amidoethercarboxylates of the formula:

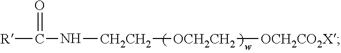

alkyl succinates of the formula:

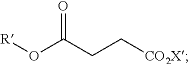

fatty acyl sarcosinates of the formula:

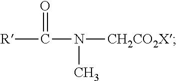

fatty acyl amino acids of the formula:

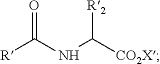

fatty acyl taurates of the formula:

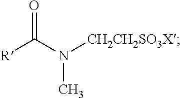

fatty alkyl sulfoacetates of the formula:

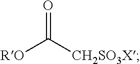

alkyl phosphates of the formula:

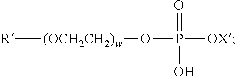

wherein
R′ is an alkyl group having from about 7 to about 22, and preferably from about 7 to about 16 carbon atoms,
R′₁ is an alkyl group having from about 1 to about 18, and preferably from about 8 to about 14 carbon atoms,
R′₂ is a substituent of a natural or synthetic I-amino acid,
X′ is selected from the group consisting of alkali metal ions, alkaline earth metal ions, ammonium ions, and ammonium ions substituted with from about 1 to about 3 substituents, each of the substituents may be the same or different and are selected from the group consisting of alkyl groups having from 1 to 4 carbon atoms and hydroxyalkyl groups having from about 2 to about 4 carbon atoms and
v is an integer from 1 to 6;
w is an integer from 0 to 20; and mixtures thereof.

According to certain embodiments, the anionic surfactant of the present invention preferably comprises one or more alkyl ether sulfates, or mixtures thereof. In certain more preferred embodiments, the anionic surfactant of the present invention comprises sodium trideceth sulfate. Sodium trideceth sulfate is the sodium salt of sulfated ethoxylated tridecyl alcohol that conforms generally to the following formula, C₁₃H₂₇ (OCH₂CH₂)ₙOSO₃Na, where n has a value between 1 and 4, and is commercially available from Stepan Company of Northfield, Ill. under the tradename, “Cedapal TD-403M.”

As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions.

Examples of amphoteric surfactants suitable for use in the present invention include, but are not limited to, amphocarboxylates such as alkylamphoacetates (mono or di); alkyl betaines; amidoalkyl betaines; amidoalkyl sultaines; amphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

Examples of suitable amphocarboxylate compounds include those of the formula:

A-CONH(CH₂)ₓN⁺R₅R₆R₇
wherein
A is an alkyl or alkenyl group having from about 7 to about 21, e.g. from about 10 to about 16 carbon atoms;
x is an integer of from about 2 to about 6;
R₅ is hydrogen or a carboxyalkyl group containing from about 2 to about 3 carbon atoms;
R₆ is a hydroxyalkyl group containing from about 2 to about 3 carbon atoms or is a group of the formula:

R₈—O—(CH₂)ₙCO₂⁻
wherein
R₈ is an alkylene group having from about 2 to about 3 carbon atoms and n is 1 or 2; and
R₇ is a carboxyalkyl group containing from about 2 to about 3 carbon atoms;

Examples of suitable alkyl betaines include those compounds of the formula:

B—N⁺R₉R₁₀(CH₂)ₚCO₂⁻
wherein
   B is an alkyl or alkenyl group having from about 8 to about 22, e.g., from about 8 to about 16 carbon atoms;
   R₉ and R₁₀ are each independently an alkyl or hydroxyalkyl group having from about 1 to about 4 carbon atoms; and
   p is 1 or 2.

A preferred betaine for use in the present invention is lauryl betaine, available commercially from Albright & Wilson, Ltd. of West Midlands, United Kingdom as “Empigen BB/J.”

Examples of suitable amidoalkyl betaines include those compounds of the formula:

D-CO—NH(CH₂)_{q}—N⁺R₁₁R₁₂(CH₂)ₘCO₂⁻
wherein
   D is an alkyl or alkenyl group having from about 7 to about 21, e.g. from about 7 to about 15 carbon atoms;
   R₁₁ and R₁₂ are each independently an alkyl or
   Hydroxyalkyl group having from about 1 to about 4
   carbon atoms;
   q is an integer from about 2 to about 6; and m is 1 or 2.

One amidoalkyl betaine is cocamidopropyl betaine, available commercially from Goldschmidt Chemical Corporation of Hopewell, Va. under the tradename, “Tegobetaine L7.”

Examples of suitable amidoalkyl sultaines include those compounds of the formula

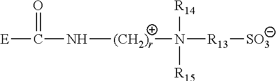

wherein
   E is an alkyl or alkenyl group having from about 7 to about 21, e.g. from about 7 to about 15 carbon atoms;
   R₁₄ and R₁₅ are each independently an alkyl, or hydroxyalkyl group having from about 1 to about 4 carbon atoms;
   r is an integer from about 2 to about 6; and
   R₁₃ is an alkylene or hydroxyalkylene group having from
   about 2 to about 3 carbon atoms;

In one embodiment, the amidoalkyl sultaine is cocamidopropyl hydroxysultaine, available commercially from Rhone-Poulenc Inc. of Cranbury, N.J. under the tradename, “Mirataine CBS.”

Examples of suitable amphophosphate compounds include those of the formula:

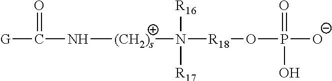

wherein
   G is an alkyl or alkenyl group having about 7 to about 21, e.g. from about 7 to about 15 carbon atoms;
      s an integer from about 2 to about 6;
      R₁₆ is hydrogen or a carboxyalkyl group containing from about 2 to about 3 carbon atoms;
      R₁₇ is a hydroxyalkyl group containing from about 2 to about 3 carbon atoms or a group of the formula:

R₁₉—O—(CH₂)ₜ—CO₂⁻
wherein R₁₉ is an alkylene or hydroxyalkylene group having from about 2 to about 3 carbon atoms and t is 1 or 2; and
R₁₈ is an alkylene or hydroxyalkylene group having from about 2 to about 3 carbon atoms.

In one embodiment, the amphophosphate compounds are sodium lauroampho PG-acetate phosphate, available commercially from Mona Industries of Paterson, N.J. under the tradename, “Monateric 1023,” and those disclosed in U.S. Pat. No. 4,380,637, which is incorporated herein by reference.

Examples of suitable phosphobetaines include those compounds of the formula:

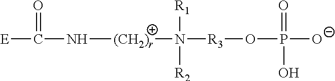

wherein E, r, R₁, R₂ and R₃, are as defined above. In one embodiment, the phosphobetaine compounds are those disclosed in U.S. Pat. Nos. 4,215,064, 4,617,414, and 4,233,192, which are all incorporated herein by reference.

Examples of suitable pyrophosphobetaines include those compounds of the formula:

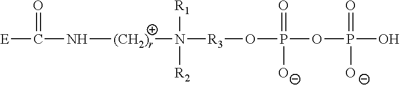

wherein E, r, R₁, R₂ and R₃, are as defined above. In one embodiment, the pyrophosphobetaine compounds are those disclosed in U.S. Pat. Nos. 4,382,036, 4,372,869, and 4,617,414, which are all incorporated herein by reference.
Examples of suitable carboxyalkyl alkylpolyamines include those of the formula:

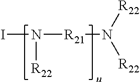

wherein
   I is an alkyl or alkenyl group containing from about 8 to about 22, e.g. from about 8 to about 16 carbon atoms;
   R₂₂ is a carboxyalkyl group having from about 2 to about 3 carbon atoms;
   R₂₁ is an alkylene group having from about 2 to about 3 carbon atoms and
   u is an integer from about 1 to about 4.

Any suitable amounts of polymeric material and surfactants may be used in accord with the present invention. In certain preferred embodiments, the present composition comprise from greater than zero to about 6 weight percent of polymeric material (based on active amount of polymeric material in the total weight of composition). In certain more preferred embodiments, the compositions comprise from about 0.1 to about 4.5 weight percent of polymeric material, more preferably from about 0.1 to about 3.5 weight percent of polymeric material, and even more preferably from about 0.2 to about 2 weight percent of polymeric material. In certain preferred embodiments, the present composition comprise from greater than zero to about 15 weight percent of anionic and amphoteric surfactants (or amphoteric surfactant alone) (based on total active amount of anionic and amphoteric surfactant(s) in the total weight of composition). In certain more preferred embodiments, the compositions comprise from about 1 to about 12 weight percent of anionic and amphoteric surfactants, more preferably from about 4 to about 11 weight percent of anionic and amphoteric surfactants, and even more preferably from about 5 to about 10.5 weight percent of anionic and amphoteric surfactant. In certain more preferred embodiments, the compositions comprise from about zero to about 4.5 weight percent of anionic surfactant, more preferably from about 0 to about 3.5 weight percent of anionic surfactant, and even more preferably from about 0.2 to about 2 weight percent of anionic surfactant.

In certain more preferred embodiments, the compositions contain from about 1 to about 12 weight percent of amphoteric surfactant and are largely devoid of anionic surfactant, more preferably from about 2 to about 10 weight percent of amphoteric surfactant and largely devoid of anionic surfactant, and even more preferably from about 2.5 to about 9 weight percent of amphoteric surfactant and largely devoid of anionic surfactant.

The polymeric material and anionic/amphoteric surfactant may be combined according to the present invention via any conventional methods of combining two or more fluids. For example, one or more compositions comprising, consisting essentially of, or consisting of at least one polymeric material and one or more compositions comprising, consisting essentially of, or consisting of at least one anionic and/or amphoteric surfactant may be combined by pouring, mixing, adding dropwise, pipetting, pumping, and the like, one of the compositions comprising polymeric material or surfactant into or with the other in any order using any conventional equipment such as a mechanically stirred propeller, paddle, and the like. According to certain embodiments, the combining step comprises combining a composition comprising anionic and/or amphoteric surfactant into or with a composition comprising polymeric material. According to certain other embodiments, the combining step comprises combining a composition comprising polymeric material into or with a composition comprising anionic and/or amphoteric surfactant.

The cleansing compositions produced, as well as any of the compositions comprising polymeric material or anionic and/or amphoteric surfactant that are combined in the combining step according to the present methods may further comprise any of a variety of other components nonexclusively including one or more nonionic and/or cationic surfactants, pearlescent or opacifying agents, thickening agents, secondary conditioners, humectants, chelating agents, and additives which enhance the appearance, feel and fragrance of the compositions, such as colorants, fragrances, preservatives, pH adjusting agents, and the like.

Any of a variety of commercially available secondary conditioners, such as volatile silicones, which impart additional attributes, such as gloss to the hair are suitable for use in this invention. In one embodiment, the volatile silicone conditioning agent has an atmospheric pressure boiling point less than about 220° C. The volatile silicone conditioner may be present in an amount of from about 0 percent to about 3 percent, e.g. from about 0.25 percent to about 2.5 percent or from about 0.5 percent to about 1 percent, based on the overall weight of the composition. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Mich. under the tradename, “DC-345” and mixtures thereof, and preferably include cyclomethicone fluids.

A variety of commercially available humectants, which are capable of providing moisturization and conditioning properties to the personal cleansing composition, are suitable for use in the present invention. The humectant may be present in an amount of from about 0 percent to about 10 percent, e.g. from about 0.5 percent to about 5 percent or from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, dipropylene glycol, and mixtures thereof; 2) polyalkylene glycol of the formula: HO—(R″O)_{b}—H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)_{c}—OH, wherein c is an integer from about 5 to about 25; 4) urea; and 5) mixtures thereof, with glycerine being the preferred humectant.

Examples of suitable chelating agents include those which are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Mich. under the tradename, “Versene 100XL” and is present in an amount, based upon the total weight of the composition, from about 0 to about 0.5 percent or from about 0.05 percent to about 0.25 percent.

Suitable preservatives include Quaternium-15, available commercially as “Dowicil 200” from the Dow Chemical Corporation of Midland, Mich., and are present in the composition in an amount, based upon the total weight of the composition, from about 0 to about 0.2 percent or from about 0.05 percent to about 0.1 percent.

The methods of the present invention may further comprise any of a variety of steps for mixing or introducing one or more of the optional components described hereinabove with or into a composition comprising a polymeric material and/or an anionic and/or amphoteric surfactant before, after, or simultaneously with the combining step described above. While in certain embodiments, the order of mixing is not critical, it is preferable, in other embodiments, to pre-blend certain components, such as the fragrance and the nonionic surfactant before adding such components into a composition comprising a polymeric material and/or an anionic surfactant.

The cleansing methods of the present invention may further comprise any of a variety of additional, optional steps associated conventionally with cleansing hair and skin including, for example, lathering, rinsing steps, and the like.

Although applicants do not wish to be bound by or to any particular theory of operation, it is believed that surfactant associated with the low molecular weight hydrophobically-modified polymer is more stable than surfactants that exist as a micelle. Thus, surfactant contained in a micelle structure more readily disperses out of the micelle than it does when associated with low molecular weight hydrophobically-modified polymer.

The foregoing information regarding low molecular weight hydrophobically-modified polymers as well as compositions that may be useful in the methods of this invention are set forth in US2008/0112913, US2006/0257348, and US20070111910, all of which are hereby incorporated herein by reference.

The methods and compositions of this invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### Example 1

### Exaggerated Arm Wash Study—Skin with Impaired Barrier Due to Soap-Induced Xerosis

This Example 1 relates to the study of the effect of cleansing skin with impaired barrier utilizing both compositions of this invention and with comparative compositions.

Two exaggerated arm wash studies were conducted independently and on two different sets of subjects. Each study was carried out at a different time of the year, one in winter and one in spring. In the studies, the skin of normal adults (aged 22-55) with no history of skin condition was impaired with repeated exposure to bar soap (Ivory® Proctor & Gamble, Cincinnati, Ohio). The ingredient of Ivory™ soap is listed as follows: Sodium Tallowate, Sodium Palmate, Water, Sodium Cocoate, Sodium Palm Kernelate, Glycerin, Sodium Chloride, Fragrance, Coconut Acid, Palm Kernel Acid, Tallow Acid, Palm Acid, Tetrasodium EDTA. The subjects (n=9-10 subjects/treatment cell) cleansed their lower volar forearms twice a day for seven days with Ivory soap. No lotion was applied to their skin. This treatment led to an increase in TEWL, thus an impaired barrier function of the cleansed skin, and a decreased level of skin hydration. The TEWL value was measured for each subject using a Vapometer (Delfin Technologies, Finland) by holding a probe on the skin surface for a period of time. The probe was connected to a computer or other data recording device. The probe was designed to measure relative humidity at two points above the skin surface. Water flux (TEWL) was then determined from the relative humidity levels. TEWL was expressed as mg/cm²/hr (or g/m²/h).

TEWL and hydration measurements were conducted at Day 0 (before soap dry-out), Day 1 (Baseline, after 1 week dry-out with soap), Day 3 (3 days of washing with the test cleanser), and Day 5 (3 days of washing with the test cleanser). The results are reported as a change from the Day 1 baseline (i.e. TEWL or Hydration Day 5—TEWL or hydration Day 1). After the soap treatment at Day 1, the skin was less hydrated and the barrier was impaired, as evidenced by the decrease in conductance and the increase in TEWL from Day 0 to Day 1 measurements.

This is representative of soap-induced xerosis of the skin. In this impaired barrier state, the hydration of the skin was lowered, and the measurement of the skin barrier via TEWL was significantly higher.

During the second phase of the study, the test cleansers set forth below in Table 1 were then used to cleanse the skin and TEWL measurements taken.

### Foam Volume Test Experimental Procedure:

An industrially accepted means to measure the foam generation of the consumer product is the Sita Foam Tester R-2000 (SITA Messtechnik GmbH, Dresden Germany). Specifically designed to measure foam generation, the Sita Foam Tester consists of a jacketed sample vessel with and agitator. To represent the hard water of tap water, 0.36 g of calcium chloride is dissolved in 995 g of DI water. Five (5) grams of test formula is added to this solution and mixed until homogeneous. Then this 0.5% dilution of test formula is placed in the holding tank of the Sita Foam Tester. For each experimental run, 250 ml of solution is introduced into the test vessel and allowed to come to 30° C. ±2° C. The agitator spins at 1200 rpm for 15 seconds, then the foam volume is measured. The agitation is repeated for a total of 12 cycles. The foam generation test is conducted 3 times for each test sample.

The cleansing compositions of Examples C1, C2, E1 and E2 were prepared according to the materials and amounts listed in Table 1.

TABLE 1*

| | C1 | E1 | C2 | E2 |
|---|---|---|---|---|
| INCI Name | (wt %) | (wt %) | (wt %) | (wt %) |
| Purified Water | Q.S. | Q.S. | Q.S. | Q.S. |
| Cocamidopropyl Betaine | 3.75 | 3.75 | 3.60 | 3.75 |
| Sodium Trideceth Sulfate | 2.70 | 2.70 | 0 | 0 |
| Decyl Glucoside | 0 | 0 | 3.12 | 3.12 |
| Disodium | 0 | 0 | 0.60 | 0.60 |
| Lauroamphodiacetate | | | | |
| Potassium Acrylates | 0 | 1.80 | 0.00 | 1.80 |
| Copolymer | | | | |
| PEG-80 Sorbitan Laurate | 3.60 | 3.60 | 4.32 | 4.32 |
| Polyquaternium-10 | 0.14 | 0.14 | 0.14 | 0.14 |
| Tetrasodium EDTA | 0.25 | 0.25 | 0.25 | 0.25 |
| PEG-150 Distearate | 0.18 | 0.18 | 0.90 | 0.90 |
| Fragrance | 0.12 | 0.12 | 0.12 | 0.12 |
| Quaternium-15 | 0.05 | 0.05 | 0.05 | 0.05 |
| Sodium Chloride | Q.S. | Q.S. | Q.S. | Q.S. |
| Citric Acid | Q.S. | Q.S. | Q.S. | Q.S. |
| Sodium Hydroxide | Q.S. | Q.S. | Q.S. | Q.S. |
| Foam Test results | | | | |
| Foam Vol (ml @ 90s) | 238 | 298 | | 277 |
| Foam Vol (ml @ max) (Fₘₐₓ) | 287 | 392 | | 362 |

*expressed in active % w/w

Each of the compositions of Table 1 was independently prepared as follows: Water (50.0 parts) was added to a beaker. The following ingredients were then added thereto independently with mixing until each respective resulting mixture was homogenous: Potassium Acrylates Copolymer (hm-polymer), Cocamidopropyl Betaine (CAPB), Decyl Glucoside (if called for), PEG-80 Sorbitain Laurate, Sodium Trideceth Sulfate (TDES) (if called for) and the remainder of the ingredients. The pH of the resulting solution was then adjusted with a 20% solution of Sodium hydroxide or Citric Acid solution until the final desired pH of about 6.3 to about 6.6 was obtained. The remainder of the water was then added thereto.

Comparative Sample 3, C3, is the creamy cleanser Cetaphil® Gentle Skin Cleanser, for all skin types (Galderma). Cetaphil® is consider a gentle cleanser appropriate for patients with an impaired barrier, and is commonly recommended by dermatologist for patients with impaired skin barrier. Cetaphil's ingredients are listed as: Water, Cetyl Alcohol, Propylene Glycol, Sodium Lauryl Sulfate, Stearyl Alcohol, Methylparaben, Propylparaben, Butylparaben.

Skin hydration after exposure to the compositions set forth in Table I was measured by skin conductance (micro Siemans, “μS”). The measurement of skin hydration is set forth below in Table 2a and 2b. as a change from the skin hydration measured after cleansing with the test cleanser. Skin conductance was measured on Day 1, Day 3 and Day 5. The differences in the measurements taken on Day 1 and Day 3, and between those taken on Day 1 and Day 5 were calculated. Skin hydration was generally observed to increase over the course of the study. At Day 3 in some treatments, the skin hydration was observed to have decreased further compared to Day 1 (after Ivory™ treatment), however, this result is not statistically significantly different from skin hydration at Day 1 (after Ivory™ treatment). The lack of improvement in skin hydration at Day 3 was attributed to the slow recovery of the damaged skin barrier from soap-induced xerosis, which was induced by cleansing for 5 days with soap.

After five days of cleansing with a mild cleansing product, the improvement in skin hydration was readily apparent, with differing levels of improvements based on the cleansing composition. In Table 2a, skin treated with E1, the foaming cleanser with low molecular weight hm-polymer, Potassium Acrylates Copolymer, was observed to increase the skin hydration to a greater level as compared to C1 which did not contain the low molecular weight hm-polymer, Potassium Acrylates Copolymer. The above results suggest that E1 allows the skin to heal more rapidly.

The response of the skin hydration to treatment with E1 (containing low molecular weight hm-polymer) was similar to the response with C3, Cetaphil™, a non-foaming cleanser.

Similarly, in Table 2b, the foaming cleansing with low molecular weight hm-polymer, was observed to increase the skin hydration to a greater level, as compared to C2 (without low molecular weight hm-polymer). Again, suggesting that E2 allows the skin to heal more rapidly.

Skin hydration levels were also collected on a location on the arm corresponding to “Untreated” skin, which was not exposed to any cleansing treatment. This skin was considered to have a skin hydration level that is indicative of normal, healthy skin, rather than soap-induced xerosis. As set forth in Table 2b, at Day 5, the foaming cleanser with low molecular weight hm-polymer, Potassium Acrylates Copolymer, (Example 2) was observed to increase the skin hydration levels to levels equivalent to “Untreated”, indicating that the skin has returned to skin hydration levels similar to that of normal, healthy skin.

TABLE 2a

| | | Day 3-Day 1 | Day 5-Day 1 |
|---|---|---|---|
| | Cleansing | Delta Skin | Delta Skin |
| Examples | composition | conductance (μS) | conductance (μS) |
| C1 | TDES/CAPB/no | −5.1 | 7.6 |
| | hm-polymer | | |
| E1 | TDES/CAPB/with | −0.9 | 20.3 |
| | hm-polymer | | |
| C3 | Cetaphil | 13.3 | 20.7 |

TABLE 2b

| | | Day 3-Day 1 | Day 5-Day 1 |
|---|---|---|---|
| | Cleansing | Delta Skin | Delta Skin |
| Examples | composition | conductance (μS) | conductance (μS) |
| C2 | CAPB no hm- | −0.8 | 4.2 |
| | polymer | | |
| E2 | CAPB with hm- | 11.1 | 12.1 |
| | polymer | | |
| | Untreated | 6.4 | 12.9 |

Through exposure of the skin to a mild cleansing treatment of the present invention (i.e., E1 and E2), the skin was able to return back to its normal healthy state, with a return to a higher hydration state, as evidenced by the higher conductance values.

TABLE 3

| | | Day 3- | | Day 5- | |
|---|---|---|---|---|---|
| | | Day 1 | | Day 1 | |
| | | Delta | | Delta | |
| | Cleansing | TEWL | | TEWL | |
| | composition | (g/m²/hr) | | (g/m²/hr) | |
| Examples | TEWL | Ave | St Dev | Ave | St Dev |
| Study 1 | | | | | |
| C1 | TDES/CAPB/ | 0.2 | 1.6 | 0.4 | 1.5 |
| | no hm-polymer | | | | |
| E1 | TDES/CAPB/ | −1.2 | 1.7 | −0.7 | 1.4 |
| | with hm- | | | | |
| | polymer | | | | |
| C3 | Cetaphil | −0.1 | 1.1 | −0.2 | 0.9 |
| Study 2 | | | | | |
| C2 | CADB no hm- | 0.5 | 1.7 | 1.7 | 2.4 |
| | polymer | | | | |
| E2 | CAPB with hm- | 0.7 | 1.4 | 1.3 | 1.4 |
| | polymer | | | | |
| | Untreated | 0.2 | 2.8 | 1.5 | 1.6 |

As set forth in Table 3 above, the results show the change from baseline TEWL measurements on Day 3 and Day 5. Under these treatment conditions and the resolution of the instrumentation, there were no measurable changes in skin barrier function, as demonstrated by the small delta TEWL values shown above.

### Example 2

### Patch Test—Skin With Impaired Barrier Due to Atopic Dermatitis

In this example, adult subjects (aged 18-65) who were diagnosed with atopic dermatitis, and therefore had skin with impaired barrier function,

(n=25 subjects/cell) were exposed to cleansing solutions set forth above in Table 1 (diluted as defined below) on their volar forearms under an occlusive patch for 24 hours. After 24 hr, the patch and cleansing solution was removed and a new patch with the same treatment was reapplied, and this was repeated for a total of four days. The skin barrier function was measured in accordance with the measurement of TEWL before treatment and then after the 4 days of patching. The TEWL reported was the change in the TEWL at Day 4 from baseline (i.e., before patching of the cleansing composition).

The data set forth in Tables 5a and 5b below demonstrate the change in TEWL (Delta TEWL), was assessed at Day 4, in comparison to Day 1, before exposure to 50% dilution in distilled water of the cleansing composition.

The cleansing compositions of Examples C9 and E9 were prepared according to the materials and amounts listed in Table 4.

TABLE 4*

| | C9 | E9 |
|---|---|---|
| | Conc | Conc |
| INCI Name | (wt %) | (wt %) |
| Cocamidopropyl Betaine | 5.4 | 5.4 |
| Sodium Laureth Sulfate | 4.5 | 4.5 |
| Guar Hydroxypropyl | 0.5 | 0.5 |
| triamonium Chloride | | |
| Glycerin | 6.0 | 6.0 |
| Tetrasodium EDTA | 0.8 | 0.8 |
| Quaternium-15 | 0.05 | 0.05 |
| Polyquaternium 10 | 0.2 | 0.2 |
| Sodium Hydroxide | Q.S. | Q.S. |
| Citric Acid | Q.S. | Q.S. |
| Water | Q.S. | Q.S. |
| Potassium Acrylates | 0 | 2.4 |
| Copolymer | | |

*expressed in active % w/w

Each of the compositions of Table 4 was independently prepared as follows: Water (50.0 parts) was added to a beaker. The following ingredients were then added thereto independently with mixing until each respective resulting mixture was homogenous: Potassium Acrylates Copolymer, Sodium Laureth Sulfate, Cocamidopropyl Betaine, and the remainder of the ingredients. The pH of the resulting solution was then adjusted with a 20% solution of Citric Acid of Sodium hydroxide solution until the final desired pH of about 6.3 to about 6.6 was obtained. The remainder of the water was then added thereto.

The inventive examples were compared with a number of commercial mild facial cleansers. C5 is Cetaphil®Daily Facial Cleanser for Normal to Oil Skin from Galderma (Fort Worth, Tex.).

C6 is Purpose® Gentle Face Cleanser from Johnson & Johnson Consumer Companies, Inc. (Skillman, N.J.). C7 is Neutrogena® Fresh Foaming Cleanser, available from Neutrogena Corporation (Los Angeles, Calif.). C8 is Aveeno® Ultra-Calming Foaming Cleanser, available from Johnson & Johnson Consumer Companies, Inc. (Skillman, N.J.).

TABLE 5a

| | | DELTA |
|---|---|---|
| | | TEWL (Day 4- |
| | | Baseline) |
| | Cleansing composition | (g/m²/hr) |
| C1 | TDES/CAPB no hm-polymer | 4.80 |
| E1 | TDES/CAPB with hm-polymer | 3.78 |
| | Water | 1.83 |
| | No treatment (patch only) | 0.23 |

TABLE 5b

| | | DELTA |
|---|---|---|
| | | TEWL (Day 4- |
| | | Baseline) |
| | Cleansing composition | (g/m²/hr) |
| C5 | Cetaphil Daily Facial Cleanser | 8.1 |
| C6 | Purpose Gentle Cleanser | 7.2 |
| C7 | Neutrogena Fresh Foaming Cleanser | 6.3 |
| C8 | Aveeno Ultra-Calming Foaming Cleanser | 5.2 |
| E2 | CAPB with hm-polymer | 2.3 |
| | Water | 1.7 |
| | No treatment (patch only) | 0.9 |

As set forth in Tables 5a and 5b, the results demonstrate that exposure to a blank patch without any cleansing composition or water alone resulted in a very small increase in TEWL, thus a small Delta TEWL value, demonstrating very minimal interaction between the test product and the skin. For cleansing compositions that are mild to the skin, the Delta TEWL was also small, indicating minimal interaction of the cleansing composition with the skin. Such mild compositions did not exacerbate the impairment of the skin barrier due to the atopic dermatitis.

In Table 5a, the foaming cleansing composition (E1) with low molecular weight hm-polymer, was observed to have a smaller Delta TEWL as compared to Comparative 1 (without low molecular weight hm-polymer). In addition, another foaming cleanser (E2) with low molecular weight hm-polymer was also observed to have a small Delta TEWL. As set forth therein, the ΔTEWL was lower when both surfactant and low molecular weight hm-polymer were present in the composition, indicating less impairment of the skin barrier using compositions in the methods of this invention.

The data set forth in Tables 6 below demonstrate the change in TEWL (Delta TEWL), was assessed at Day 4, in comparison to Day 1, before exposure to 5% dilution in distilled water of the cleansing composition.

TABLE 6

| | | DELTA |
|---|---|---|
| | | TEWL (Day 4- |
| | | Baseline) |
| | Cleansing composition | (g/m²/hr) |
| C9 | SLES/CAPB no hm-polymer | 9.0 |
| E9 | SLES/CAPB w/ hm-polymer | 6.6 |
| | Water | 2.4 |
| | No treatment (patch only) | 1.7 |

In Table 6, the foaming cleansing composition (E9) with low molecular weight hm-polymer, was observed to have a smaller Delta TEWL as compared to Comparative 9 (without low molecular weight hm-polymer). As set forth therein, the ΔTEWL was lower when both surfactant and low molecular weight hm-polymer were present in the composition, indicating less impairment of the skin barrier using compositions in the methods of this invention.

### Example 3

### Measuring Surfactant Dynamics

### Dynamic Surface Tension Test

A drop shape analysis (DSA) system DSA100 (KRÜSS GmbH, Hamburg Germany) was used to measure the dynamic surface tensions of the surfactant solutions. The system consists of a high-speed camera, light source, syringe dosing system, and environmental chamber for humidity control to reduce drop evaporation. Pendant drops of surfactant solution were formed at the tip of an inverted needle of 1.853 mm outer diameter. Drop sizes ranged from 10 to 25 μL and surfactant concentrations of 288 mg/L and 719 mg/L were investigated for each surfactant mixture. A high-speed camera acquired images at a frame rate of 6.25 frame/s for the first 10 s, 2.08 frame/s for the next 50 s, and 1.09 frame/s for the last 60 s. DSA1 image analysis software defines the drop edge can then be used to calculate the surface tensions for each drop image by the Laplace-Young equation using the x- and z-axis radii of the drop outline.

### Equilibrium Tensiometry Test

A method to measure the equilibrium surface tension, γ_{eq}, of surfactant solutions is the Wilhelmy plate method (Holmberg, K.; Jonsson, B.; Kronberg, B.; Lindman, B. Surfactants and Polymers in Aqueous Solution, Wiley & Sons, p. 347). In this method, a plate is submerged into a liquid and the downward force exerted by of the liquid on the plate is measured. The surface tension of the liquid can then be determined based on the force on the plate and the dimensions of the plate. By measuring the surface tension over a range of concentrations the critical micelle concentration (CMC) can then be determined.

In the following examples, a Kruss K100 Tensiometer (Kruss USA, Mathews, N.C.) with a platinum Wilhelmy plate used to determine the equilibrium surface tension of each sample over a range of concentrations. A sample vessel contains some initial solution in which the Wilhelmy plate measures the surface tension. Then a second solution is dosed into the sample vessel, stirred, and then probed again with the Wilhelmy plate.

The cleansing compositions of Examples and comparative samples C10 through C14 and E10 through E14 were prepared according to the materials and amounts listed in Table 7

TABLE 7

| | E10 | C10 | E11 | E12 | C11 | E13 | C13 |
|---|---|---|---|---|---|---|---|
| | Conc | Conc | Conc | Conc | Conc | Conc | Conc |
| INCI Name | (wt %) | (wt %) | (wt %) | (wt %) | (wt %) | (wt %) | (wt %) |
| Cocamidopropyl | 0.29 | 0.29 | 0.58 | 0.58 | 0.58 | 0 | 0 |
| Betaine | | | | | | | |
| Sodium Trideceth | 0.29 | 0.29 | 0 | 0 | 0 | 0.58 | 0.58 |
| Sulfate | | | | | | | |
| Potassium Acrylates | 0.096 | 0 | 0.096 | 0.048 | 0 | 0.096 | 0 |
| Copolymer | | | | | | | |
| Water | q.s. | q.s. | q.s. | q.s. | q.s. | q.s. | q.s. |

| | E14 | C14 |
|---|---|---|
| | Conc | Conc |
| INCI Name | (wt %) | (wt %) |
| Sodium Methyl 2-Sulfolaurate | 0.29 | 0.29 |
| Disodium 2-Sulfolaurate | 0.29 | 0.29 |
| Potassium Acrylates Copolymer | 0.096 | 0 |
| Water | q.s. | q.s. |

*expressed in active % w/w

Each of the compositions of Table 7 was independently prepared as follows: Water (50.0 parts) was added to a beaker. The following ingredients were then added thereto independently with mixing until each respective resulting mixture was homogenous: Potassium Acrylates Copolymer, Sodium Trideceth Sulfate, Cocamidopropyl Betaine, Sodium Methyl 2-Sulfolaurate (SMSl), Disodium 2-Sulfolaurate (dSSl). The pH of the resulting solution was then adjusted with a 20% solution of Citric Acid or Sodium hydroxide solution until the final desired pH of about 6.3 to about 6.6 was obtained. The remainder of the water was then added thereto.

TABLE 8
ΔST(t) and [γ(t) − y_{eq}] for samples C10-C14 and E10-
E14, calculated from the dynamic surface tension test
and the Equilibrium Tensiometry test.

| | | | | | E10 | |
|---|---|---|---|---|---|---|
| | C13 | E13 | | C10 | tdes/capb/hm- | |
| | tdes | tdes/hm-polymer | | tdes/capb | polymer | |
| time | γ(t) − γ_{eq} | γ(t) − y_{eq} | ΔST(t) | γ(t) − γ_{eq} | γ(t) − γ_{eq} | ΔST(t) |
| (s) | (mN/m) | (mN/m) | (mN/m) | (mN/m) | (mN/m) | (mN/m) |
| 1.1 | 9.6 | 11.3 | 1.7 | 9.2 | 9.8 | 0.6 |
| 2.1 | 8.3 | 10.0 | 1.7 | 8.3 | 9.2 | 0.9 |
| 3.0 | 7.4 | 9.3 | 2.0 | 7.5 | 8.6 | 1.2 |
| 5.0 | 6.1 | 8.4 | 2.3 | 6.4 | 7.8 | 1.4 |
| 6.9 | 5.3 | 7.7 | 2.4 | 5.8 | 7.3 | 1.5 |
| 9.8 | 4.4 | 7.0 | 2.6 | 5.0 | 6.7 | 1.7 |
| 15 | 3.4 | 6.0 | 2.6 | 4.2 | 6.0 | 1.8 |
| 20 | 2.6 | 5.3 | 2.7 | 3.5 | 5.4 | 1.9 |
| 37 | 1.5 | 4.3 | 2.8 | 2.6 | 4.6 | 2.1 |
| 65 | 0.7 | 3.6 | 2.9 | 1.8 | 4.1 | 2.3 |
| 85 | 0.4 | 3.3 | 2.9 | 1.6 | 3.9 | 2.3 |
| 120 | 0.0 | 2.9 | 3.0 | 1.3 | 3.7 | 2.4 |

| | | E12 | | | |
|---|---|---|---|---|---|
| | C11 | capb/hm- | | E11 | |
| | capb | polymer(.5) | | capb/hm-polymer | |
| Time | γ(t) − γ_{eq} | γ(t) − γ_{eq} | ΔST(t) | γ(t) − γ_{eq} | ΔST(t) |
| (s) | (mN/m) | (mN/m) | (mN/m) | (mN/m) | (mN/m) |
| 1.1 | 7.9 | 13.7 | 5.8 | 14.9 | 7.0 |
| 2.1 | 7.6 | 13.2 | 5.6 | 14.5 | 6.8 |
| 3.0 | 7.4 | 12.8 | 5.4 | 14.1 | 6.8 |
| 5.0 | 6.9 | 12.2 | 5.3 | 13.6 | 6.7 |
| 6.9 | 6.5 | 11.7 | 5.2 | 13.2 | 6.7 |
| 9.8 | 6.0 | 11.2 | 5.2 | 12.7 | 6.7 |
| 15 | 5.4 | 10.5 | 5.1 | 12.1 | 6.7 |
| 20 | 4.9 | 10.0 | 5.1 | 11.6 | 6.8 |
| 37 | 3.9 | 8.9 | 5.0 | 10.8 | 6.9 |
| 65 | 2.9 | 7.7 | 4.8 | 9.8 | 7.0 |
| 86 | 2.5 | 7.0 | 4.5 | 9.3 | 6.9 |
| 120 | 2.1 | 6.0 | 4.0 | 8.5 | 6.4 |

| | C14 | E14 | |
|---|---|---|---|
| | SMS1/dSS1 | SMS1/dSS1/hm-polymer | |
| Time | γ(t) − γ_{eq} | γ(t) − γ_{eq} | ΔST(t) |
| (s) | (mN/m) | (mN/m) | (mN/m) |
| 1.1 | 11.6 | 17.4 | 5.6 |
| 2.1 | 11.8 | 15.8 | 5.1 |
| 3.0 | 10.7 | 15.0 | 5.2 |
| 5.0 | 9.8 | 13.7 | 5.2 |
| 6.9 | 8.5 | 12.6 | 4.9 |
| 9.8 | 7.7 | 11.4 | 4.7 |
| 15 | 6.7 | 10.2 | 4.5 |
| 20 | 5.7 | 9.3 | 4.2 |
| 37 | 5.1 | 7.9 | 4.3 |
| 65 | 3.6 | 6.9 | 3.8 |
| 86 | 2.8 | 6.4 | 3.6 |
| 120 | 2.4 | 5.8 | 3.5 |

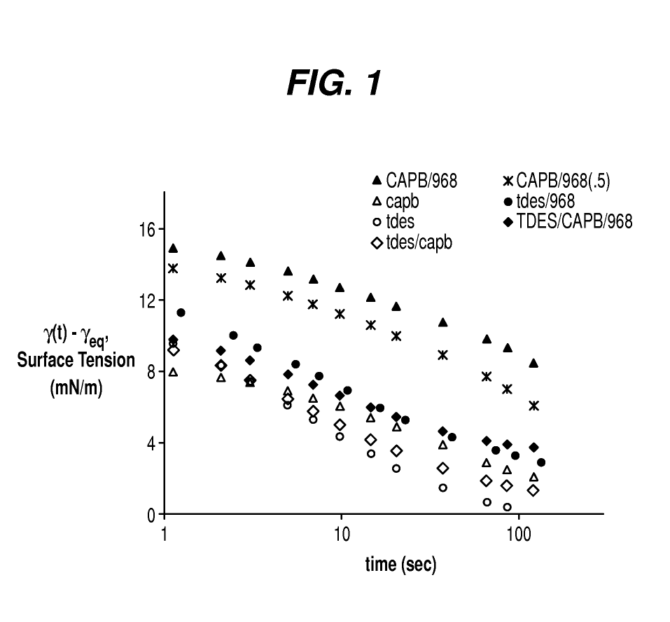

ΔST(t) and [γ(t)−γ_{eq}], as calculated from the dynamic surface tension test and the Equilibrium Tensiometry test, are shown in Table 8. [γ(t)−γ_{eq}] is also represented graphically and shown in FIG. 1. For all samples, when the new interface is formed at time=0, the deviation in the surface tension from the equilibrium surface tension, [γ(t)−γ_{eq}], is initially high (as the surface tension of the solution approaches the surface tension of pure water, 72 mN/m), then with increasing time, the [γ(t)−γ_{eq}] is observed to decrease monotonically. With increasing time, [γ(t)−γ_{eq}] decreases as γ(t) approaches γ_{eq}]. The reduction in the dynamic surface tension with time, γ(t), suggests that more surfactant is reaching the newly formed air/water interface at longer times.

As seen in FIG. 1, both C13 (containing tdes), shown as open circles, and E13 (containing tdes and low molecular weight hm-polymer), shown as closed circles, display a similar behavior of decreasing [γ(t)−γ_{eq}] with increasing time, as described above. However the decrease of [γ(t)−γ_{eq}] is slower for E13 than that of C13; the surfactant dynamics of E13 and E10 (with low molecular weight hm-polymer) are slower than C13 and C10 (without low molecular weight hm-polymer). At a given time, E13 (with low molecular weight hm-polymer) has a higher surface tension, which results in a greater [γ(t) −γ_{eq}] than the comparative C13 (without low molecular weight hm-polymer). This higher [γ(t)−γ_{eq}] suggests that E13 (tdes with low molecular weight hm-polymer) has slower surfactant dynamics and less surfactant arriving at the air/water interface than C13 (containing tdes without low molecular weight hm-polymer). Cleansing compositions containing low molecular weight hm-polymer have slower surfactant dynamics than their related comparative examples. In use, a cleanser with slower surfactant dynamics will likewise have less surfactant arriving at the water-skin interface at a given point in time.

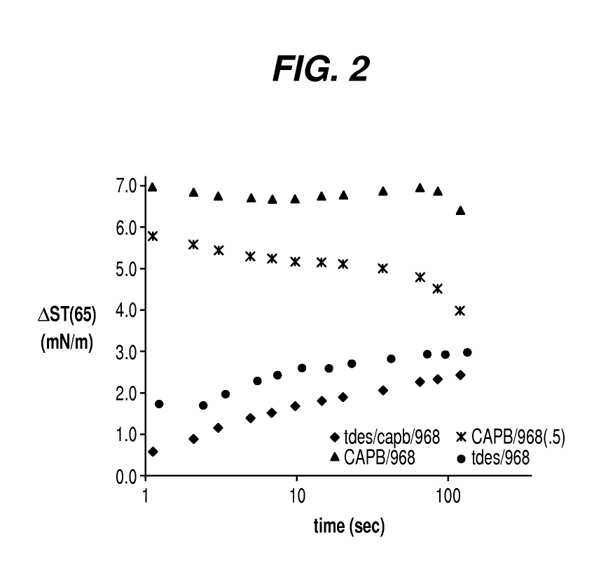

The difference in the dynamic surface tension between cleansing with a low molecular weight hm-polymer as a function of time, and a cleansing solution without hm-polymer, ΔST(t) is listed in Table 10. The ΔST(t) for E10 through E14 is displayed in FIG. 2. For all samples, E10 through E14, the ΔST(t) is positive for all times investigated, indicating that the cleansing systems with low molecular weight hm-polymer have slower surfactant dynamics then the corresponding cleansing system without low molecular weight hm-polymer.

A summary of the dynamics surface tension results at a time of 65 seconds is shown in Table 9. Again with the addition of low molecular weight hm-polymer to the cleansing system, the ΔST(65) increases, suggesting that the surfactant dynamics are slowed down.

In this dynamic surface tension test, the surfactant is observed to be slower arriving at the air/water interface with the addition of the low molecular weight hm-polymer. Correspondingly, these results also suggest the surfactant is also slower to get to the skin/water interface with the addition of low molecular weight hm-polymer. This slow down in the surfactant dynamics of these cleansing system with the addition of low molecular weight hm-polymer, allows for the improved skin compatibility that is observed in the previous examples.

TABLE 9

| | | hm- | γ | γ | ΔST |
|---|---|---|---|---|---|
| | | polymer | (65) | (65)-γ_{eq} | (65) |
| Example | Surfactant(s) | (wt %) | (mN/m) | (mN/m) | (mN/m) |
| C10 | TDES/CAPE | 0 | 28.5 | 1.8 | na |
| E10 | TDES/CAPE | 0.096 | 30.8 | 4.1 | 2.3 |
| C11 | CAPB | 0 | 28.7 | 2.9 | na |
| E12 | CAPB | 0.048 | 33.5 | 7.7 | 4.8 |
| E11 | CAPB | 0.096 | 35.6 | 9.8 | 7.0 |
| C13 | TDES | 0 | 31.9 | 0.7 | Na |
| E13 | TDES | 0.096 | 34.9 | 3.6 | 2.9 |
| C14 | SMSl/dSSl | 0 | 33.6 | 3.1 | na |
| E14 | SMSl/dSSl | 0.096 | 37.4 | 6.9 | 3.8 |

The specification and embodiments above are presented to aid in the complete and non-limiting understanding of the invention disclosed herein. Since variations and embodiments of the invention can be made without departing from its spirit and scope, the invention resides in the claims hereinafter appended.

What is claimed is:

1. A method of cleansing skin having an impaired barrier, said method comprising: applying to said skin a cleansing composition comprising (i) at least one low molecular weight, non-cross-linked, linear acrylic copolymer and (ii) at least one surfactant selected from the group consisting of anionic surfactants, amphoteric surfactants, and combinations of two or more thereof, the cleansing composition having a ΔST(65) of greater than about 2 mN/m.

2. A method of cleansing skin having an impaired barrier, said method comprising:
a) applying to said skin a cleansing composition comprising (i) at least one low molecular weight, non-cross-linked, linear acrylic copolymer and (ii) at least one surfactant selected from the group consisting of anionic surfactants, amphoteric surfactants, and combinations of two or more thereof, the cleansing composition having a ΔST(65) of greater than about 2 mN/m;
b) rinsing said skin with water.

3. A method according to claim 1 wherein when a 50% dilution of said compositions is applied to skin having an impaired barrier for four successive 24-hour occlusive patches, and the TEWL of said skin is measured prior to exposure to said compositions and after exposure to said compositions, the ΔTEWL of said skin is less than about 4.5 mg/cm²/hr.

4. A method according to claim 1 wherein when a 5% dilution of said compositions is applied to skin having an impaired barrier for four successive 24-hour occlusive patches, and the TEWL of said skin is measured prior to exposure to said compositions and after exposure to said compositions, the ΔTEWL of said skin is less than about 8 mg/cm²/hr.

5. A method according to claim 2 wherein the steps of said method are repeated for five days and wherein the hydration of the skin is measured prior to exposure to the compositions and after five days of exposure to the compositions, whereby the hydration of the skin is increased by at least about 12 μS.

6. A method of cleansing skin having an impaired barrier, said method comprising:
applying to said skin a cleansing composition comprising (i) at least one low molecular weight, non-cross-linked, linear acrylic copolymer and (ii) at least one surfactant selected from the group consisting of anionic surfactants, amphoteric surfactants, and combinations of two or more thereof, the cleansing composition having a γ(65)−γ_{eq} of greater than about 3 mN/m.

7. A method of cleansing skin having an impaired barrier, said method comprising:
applying to said skin a cleansing composition comprising (i) at least one low molecular weight, non-cross-linked, linear acrylic copolymer and (ii) at least one surfactant selected from the group consisting of anionic surfactants, amphoteric surfactants, and combinations of two or more thereof, whereby foam is generated and wherein said
foam volume at 90 seconds is greater than about 175 mL.

8. A method according to claim 7 wherein said maximum foam volume is greater than about 225 mL.

9. A method according to claim 1 wherein said cleansing composition comprises total concentrations of anionic and amphoteric surfactants of from about 1 to about 15% by weight.

10. A method according to claim 9 wherein said concentration of amphoteric surfactant is from about 1 to about 12% by weight.

11. A method according to claim 10 wherein said concentration of amphoteric surfactant is from about 1 to about 8% by weight.

12. A method according to claim 10 wherein said concentration of amphoteric surfactant is from about 2 to about 7% by weight.

13. A method according to claim 1 wherein the total concentration of anionic surfactant and amphoteric surfactant is less than about 12% by weight of the composition.

14. A method according to claim 13 wherein said the concentration of said anionic surfactant is less than about 6% by weight of the composition.

15. A method of cleansing skin having an impaired barrier, said method comprising: applying to said skin a cleansing composition comprising (i) at least one low molecular weight, non-cross-linked, linear acrylic copolymer comprising potassium acrylates copolymer and (ii) at least one surfactant selected from the group consisting of anionic surfactants at least one of which comprises sodium trideceth sulfate and an amphoteric surfactants at least one of which comprises
cocamidopropyl betaine, and combinations of two or more thereof, the cleansing composition having a ΔST(65) of greater than about 2 mN/m.

16. A method of cleansing skin having an impaired barrier, said method comprising: applying to said skin a cleansing composition comprising (i) at least one low molecular weight, non-cross-linked, linear acrylic copolymer comprising potassium acrylates copolymer and (ii) at least one surfactant selected from the group consisting of anionic surfactants at least one of which comprises sodium laureth sulfate and an amphoteric surfactants at least one of which comprises
cocamidopropyl betaine, and combinations of two or more thereof, the cleansing composition having a ΔST(65) of greater than about 2 mN/m.

17. A method according to claim 1 wherein said low molecular weight, non-cross-linked, linear acrylic copolymer comprises potassium acrylates copolymer.

18. The method of claim 1 wherein said copolymer has a viscosity of 500 mPa·s or less at a 5 wt. % polymer solids concentration in deionized water and neutralized to pH 7 with an 18 wt. % NaOH solution.

19. The method of claim 18 wherein said first monomeric component is selected from (meth)acrylic acid, itaconic acid, citraconic acid, maleic acid, fumaric acid, crotonic acid, aconitic acid, and mixtures of two or more thereof.

20. The method of claim 18 wherein said second monomeric component is selected from a monomer represented by the formula:
CH₂═CRX
wherein R is hydrogen or methyl; X is —C(O)OR′ or —OC(O)R²; R¹ is linear or branched C₁ to C₉ alkyl; and R² is hydrogen or linear or branched C₁ to C₉ alkyl.

21. The method of claim 18 wherein said second monomeric component is selected from the group consisting of ethyl (meth)acrylate, butyl (meth)acrylate, 2-ethylhexyl (meth)acrylate, vinyl acetate, 1-methylvinyl acetate, vinyl propionate, vinyl butyrate, vinyl 2-ethylhexanoate, vinyl pivalate, vinyl neodecanoate, and mixtures of two or more thereof.

22. The method of claim 18 wherein said first monomeric component is selected from the group consisting of (meth)acrylic acid and said second monomeric component is selected from the group consisting of at least one C₁ to C₉ alkyl (meth)acrylate.

23. The method of claim 18 wherein the ratio of said first monomer component to said second monomer component ranges from about 20:80 wt. % to about 50:50 (wt./wt.) based on the total weight of the monomers in the polymerization medium.

24. A method according to claim 1 wherein said personal care composition comprises a non-crosslinked, linear acrylic copolymer derived from at least one monomeric component selected from the group consisting of α,β-ethylenically unsaturated monomers containing at least one carboxylic acid group and at least one monomeric component selected from the group consisting of α,β-ethylenically unsaturated non-acid monomers containing a C₁ to C₉ alkyl group, and at least one surfactant selected from the group consisting of anionic surfactants, amphoteric surfactants, and combinations of two or more thereof, wherein said composition has a clarity of at least about 90% transmittance and said copolymer and said at least one surfactant exhibit a C₉₀ that is greater than about 500 mg/L.
